(19) United States
(12) Patent Application Publication
(10) Pub. No.: US 2009/0248131 A1
(43) Pub. Date: Oct. 1, 2009

(54) Covered Stent and Method of Making Same

(75) Inventor: Trevor Greenan, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 12/059,541

(22) Filed: Mar. 31, 2008

(51) Int. Cl. A61F 2/06; B05D 1/18

(52) U.S. Cl. 623/1.13; 427/2.24

(57) ABSTRACT

A covered stent comprises a substrate, a stent adapted to be placed in a lumen of a human body, where the stent has portions interwoven in the substrate, and encapsulation encapsulating the substrate and the portions of the stent interwoven in the substrate and forming a tubular graft member.

### FIELD OF THE INVENTION

The invention relates to encapsulated stents suitable for placement in a human body lumen such as an artery.

### BACKGROUND OF THE INVENTION

Tubular prostheses such as stents, grafts, and stent-grafts (e.g., stents having an inner and/or outer covering comprising graft material and which may be referred to as covered stents) have been used to treat abnormalities in passageways in the human body. In vascular applications, these devices often are used to replace or bypass occluded, diseased or damaged blood vessels such as stenotic or aneurysmal vessels. For example, it is well known to use stent-grafts, which comprise biocompatible graft material (e.g., Dacron® or expanded polytetrafluoroethylene (ePTFE) or some other polymer) supported by a framework (e.g., one or more stent or stent-like structures), to treat or isolate aneurysms. The framework provides mechanical support and the graft material or liner provides a blood barrier.

Aneurysms are an abnormal widening of a duct or canal such as a blood vessel and generally appear in the form of a sac formed by the abnormal dilation of the duct or vessel wall. The abnormally dilated wall typically is weakened and susceptible to rupture. Aneurysms can occur in blood vessels such as in the abdominal aorta where the aneurysm generally extends from a location below the renal arteries distally to or toward the iliac arteries.

In treating an aneurysm with a stent-graft, the stent-graft typically is placed so that one end of the stent-graft is situated proximally or upstream of the diseased portion of the vessel and the other end of the stent-graft is situated distally or downstream of the diseased portion of the vessel. In this manner, the stent-graft extends through (spans) the aneurysmal sac and beyond the proximal and distal ends thereof to replace or bypass the weakened portion. The graft material typically forms a blood impervious lumen to facilitate endovascular exclusion of the aneurysm.

Approaches for making stent-grafts such as abdominal aortic aneurysm stent-grafts have included sewing annular metallic spring elements, which may have a sinusoidal configuration, to woven materials described above such as expanded polytetrafluoroethylene, polytetrafluoroethylene, or Dacron®fabric. Other approaches have included electrospinning the stent structure with a polymer or dip coating the stent structure with a polymer. One example of a known polymeric coated stent-graft is illustrated in FIG. 1, where covered stent 100 comprises metallic springs or undulating elements 102a, 102b, 102c and 102d, which can be referred to as stents or stent elements. In this example, stents or stent elements 102a, 102b, 102c and 102d have been treated with an electrospinning or dip coating process to form tubular polymeric graft member 104, which is adhered thereto. Although electrospinning and dip coating techniques may provide stent-grafts with lower profiles, there is a relatively low contact surface area between the stents and the polymeric material and the line of contact or adhesion between the stent and the polymeric material also may not be uniform throughout the circumference of the stent resulting in non-uniform load distribution when a force is placed on the stent-graft, which can result in delamination. The materials used also can be a factor in creating a tendency for the polymeric membrane and stent to delaminate. An example of separation or detachment between the stent wire and polymeric membrane is diagrammatically depicted and designated with reference character D1 in FIG. 1. Delamination typically is more of a concern in areas of higher stress loading such as the connections between the polymeric graft material and bare spring that extend beyond the edge of the polymeric graft material as shown in FIG. 2. FIG. 2 illustrates a covered stent 110 having a known construction including a plurality of metallic undulating annular stent elements 112a, 112b, 112c . . . 112n and annular undulating bare wire spring 116. A known electrospinning or dip coating process is used to provide the stent elements with a tubular polymeric graft 114 and to secure the apexes at one end of bare spring 116 to the graft. In this example, the apexes, which can be subjected to higher stresses than the stent elements, can detach as diagrammatically shown for example with reference character D2.

There remains a need to develop and/or improve stent-graft constructions.

### SUMMARY OF THE INVENTION

The present invention involves improvements in covered stent construction.

In one embodiment according to the invention, a covered stent suitable for placement in a lumen in a human body (e.g., an artery) comprises a substrate; a tubular stent adapted to be placed in a lumen of a human body, the stent having portions interwoven in the substrate; and encapsulation encapsulating the substrate and the portions of the stent interwoven in the and forming tubular graft member.

In another embodiment according to the invention, a covered stent suitable for placement in a lumen in a human body comprises a substrate; a plurality of stents adapted to be placed in a lumen of a human body, the stents having portions interwoven in the substrate; and a tubular polymeric member covering the stents and extending through at least a portion of the substrate.

In another embodiment according to the invention, a method of making a covered stent comprises interweaving a wire though a substrate to form a tubular member; and encapsulating the tubular member to form a covered stent having a tubular cover.

The above is a brief description of some deficiencies in the prior art and advantages of embodiments according to the present invention. Other features, advantages, and embodiments according to the present invention will be apparent to those skilled in the art from the following description and accompanying drawings, wherein, for purposes of illustration only, specific embodiments are set forth in detail.

### BRIEF DESCRIPTION OF THE DRAWINGS

FIG. 1 illustrates a known covered stent configuration.

FIG. 2 illustrates a portion of another known covered stent configuration.

FIG. 3A is a cut away view of a covered stent embodiment according to the invention with a portion of the cover removed to show a stent element and a substrate in which it is integrated.

FIG. 3B1 is a sectional view taken along line 3B1-3B1 in FIG. 3A and diagrammatically illustrating locations in the tubular substrate where the annular stent is interwoven.

FIG. 3B2 is a sectional view taken through a portion of one of the stents of FIG. 3A showing the substrate and cover material

FIG. 3C is a close up view illustrating a variation of the substrate-stent configuration shown in FIG. 3A.

FIG. 3D is a close up view illustrating another variation of the substrate-stent configuration shown in FIG. 3A.

FIG. 4 is a close up view illustrating a stent-graft substrate and a bare wire spring integrated therein according to another embodiment of the invention.

FIG. 5A illustrates a known anchoring mechanism.

FIG. 5B illustrates a portion of a covered stent with the anchoring mechanism of FIG. 5A integrated therein.

FIG. 6 is a partial sectional view of a portion of a covered stent with an anchor element secured thereto.

FIG. 7 is a partial sectional view of a portion of a covered stent with an anchor element secured thereto.

FIGS. 8A and 8B illustrate assembly of another covered stent embodiment according to the invention, where FIG. 8A illustrates an anchoring attachment component for the covered stent and FIG. 8B illustrates the anchoring attachment component integrally formed in a covered stent according to another embodiment.

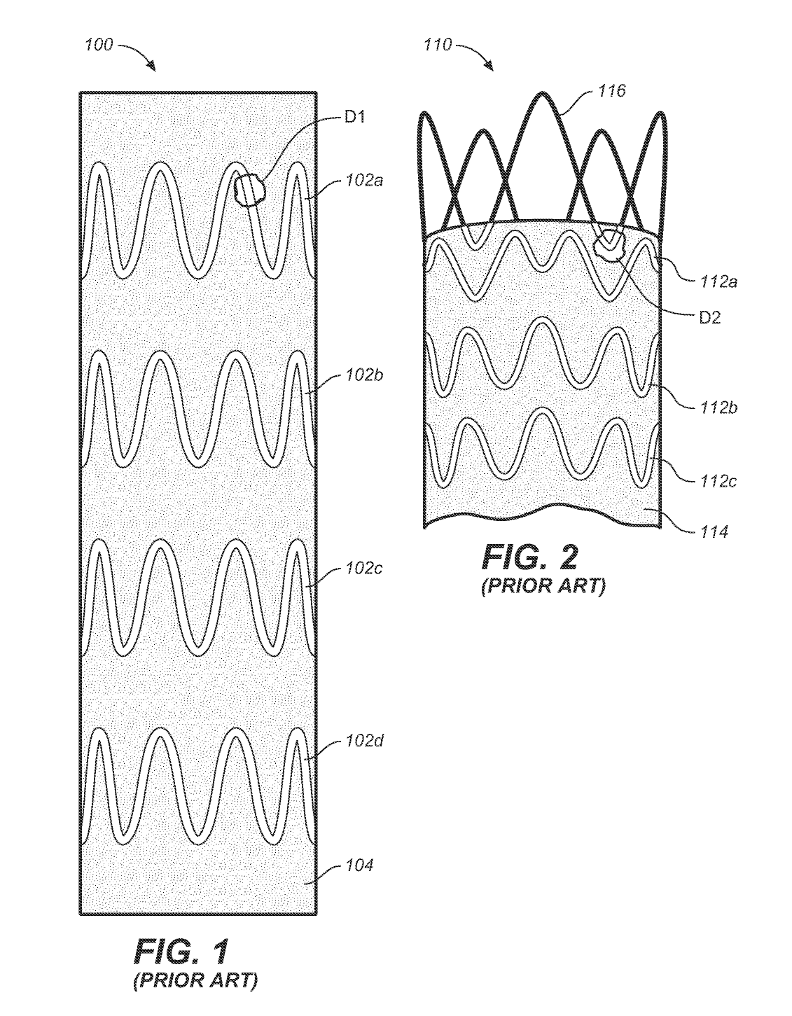

### DETAILED DESCRIPTION

The following description will be made with reference to the drawings where when referring to the various figures, it should be understood that like numerals or characters indicate like elements. Further, when referring to catheters, delivery devices, and loaded fasteners described below, the proximal end is the end nearest the operator and the distal end is farthest from the operator.

The invention generally involves a method of incorporating one or more stent and/or anchor structures into a polymeric membrane, which can be formed, for example, through known electrospinning or dip coating techniques. The stent or stents or at least a portion thereof are integrally incorporated into a material, which can be referred to as a substrate, and the integrated construction partially or wholly encapsulated (e.g., in a polymer such as polyurethane) by known electrospinning or dip coating techniques.

According to one embodiment, the substrate material can be loose textile mesh constructed from an open weave, knit, or braid. The textile mesh should be sufficiently open to easily allow the polymer to flow through it during the electrospinning or dip coating process. In the case of wire stents, incorporating or integrating the stent into the substrate material can be accomplished by passing an end of the stent wire back and forth through mesh in an annular direction and then crimping the free ends of the wire together to form an annular stent element. All of the wire need not be weaved in and out of the mesh as will be described in more detail below. Other methods of integrating the stent and mesh material include weaving or knitting the mesh around the entire stent or at least a portion of the stent. The integrated stent-substrate construction is then encapsulated or coated using, for example, a known stent electrospinning or dip coating process, to form the polymeric membrane stent cover. The stent cover provides a fluid barrier that is suitable as for use as a graft in a lumen in a human patient. Typically the cover will provide a continuous blood impervious surface suitable for use in an artery in a human patient.

One of the many advantages of this construction is that if the stent becomes completely delaminated from the polymeric membrane formed with the foregoing process, it remains attached by the polymer mesh (polymer mesh is a loose weave, braid, or knit. Further, the substrate or mesh can better distribute the load of the stent through the membrane as compared to a stent without such a substrate. The polymeric material also can be selected to improve the bonding or adhesion between the stent-substrate and the polymeric material.

The mesh substrate has a loose construction suitable for weaving the stent wire in and out of the substrate. It can be a very open weave, knit or braid. Knitted meshes typically offer more flexibility and weaves typically offer more dimensional stability. The substrate mesh can be made from a variety of materials including polyester, UHMWPE, liquid crystal polymers, and Kevlar.

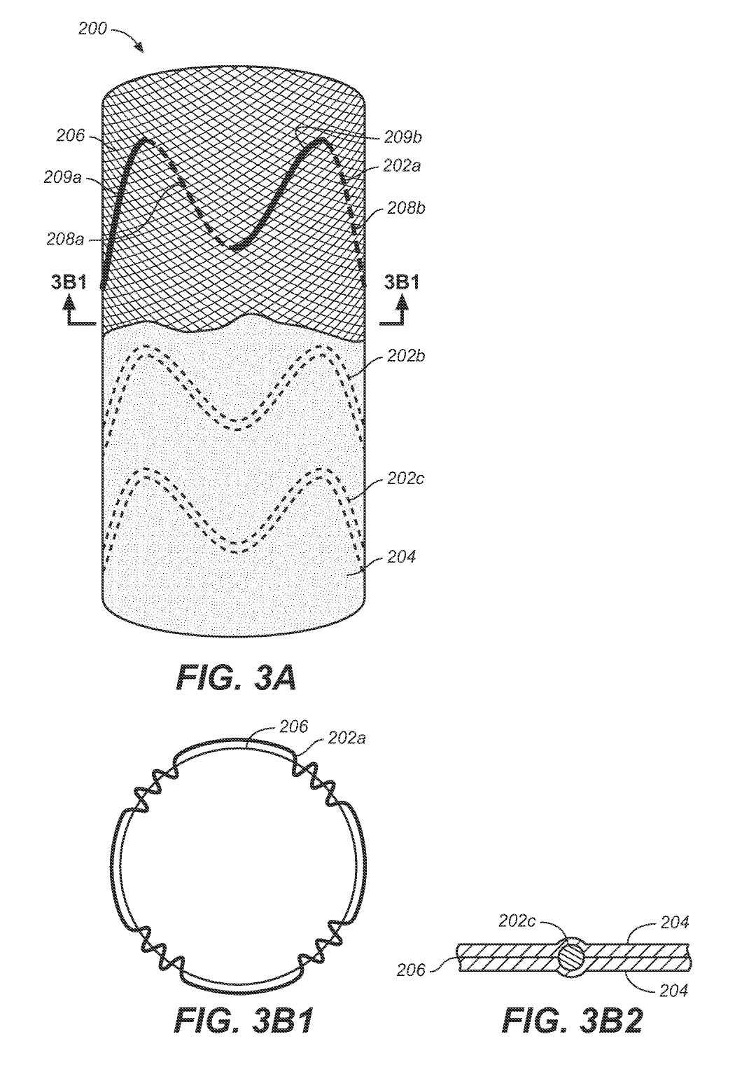

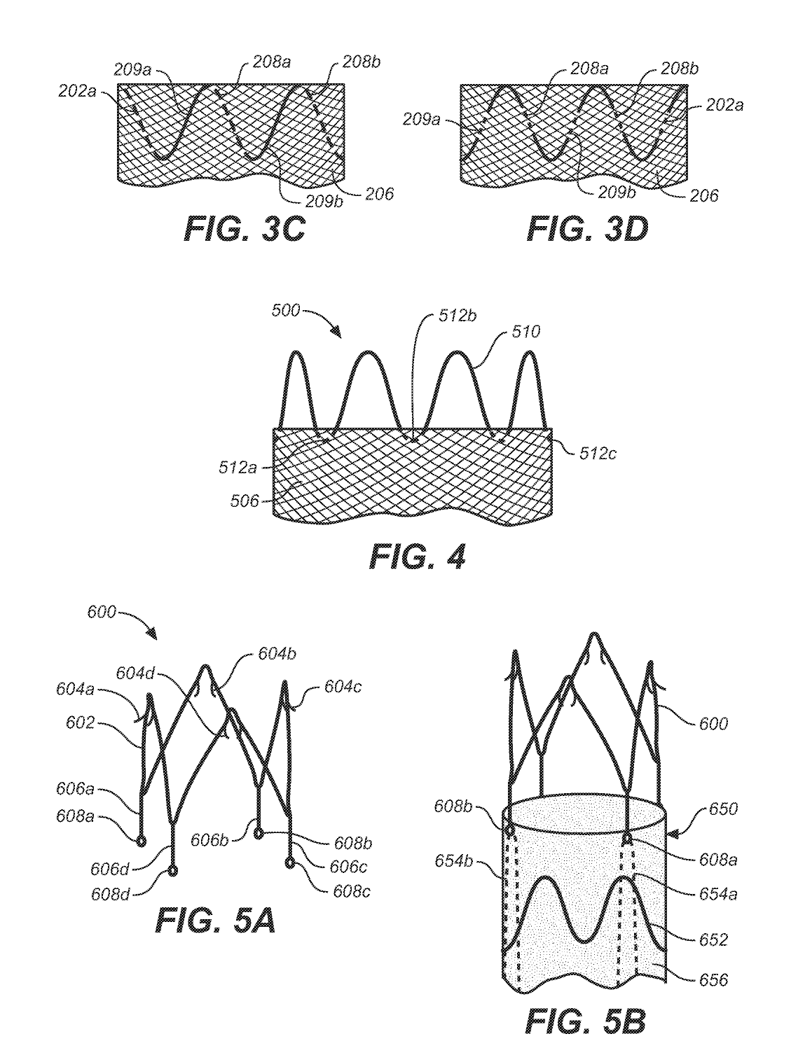

Referring to FIG. 3A, a covered stent according to one embodiment of the invention is shown in cut away view so that the substrate can be seen. In the illustrative example, covered stent 200 is shown with three stents 202a, 202b and 202c. It should be understood, however, that more or fewer stents can be used. The stents are shown with undulating annular configurations where each undulation has two leg portions which converge at an apex. The number of undulations can vary depending on the size of the stent and the application and in this example there are four undulations where two are hidden from view. In this embodiment, one of each of the two leg portions (of the undulating stent) is interwoven into the material and the other is not woven into the material and is adjacent to the inner surface or outer surface of mesh substrate 206. FIG. 3A depicts descending legs 208a and 208b interwoven in mesh substrate 206 in which appears as dashed lines, while ascending legs 209a and 209b are shown positioned along the outer surface of the substrate. The other undulating portions are similarly integrated with mesh substrate 206, but hidden from view. Thus, every other leg is interwoven into the substrate when moving in an annular or circumferential direction as diagrammatically shown in FIG. 3B1. In the variation shown in FIG. 3C, an oppositely configured pattern is used. Legs 209a, 209b, and 209c are interwoven into mesh substrate 206, while legs 208a, 208b, 208c are outside the mesh substrate prior to electrospinning or dip coating (the remaining legs forming the annular stent are hidden from view. In the variation shown in FIG. 3D, all legs of each undulation are interwoven into the mesh substrate before electrospinning or dip coating (five legs are shown in this figure with the remaining legs forming the annular stent being hidden from view).

Referring to FIG. 3B2, a sectional view of a portion of stent 200 is shown depicting stent wire 202c between two layers of polymeric material 204 on opposite sides of the mesh substrate. A noted above, the mesh substrate is sufficiently open so as to allow the polymer to pass therethrough during the electrospinning or dip coating process.

Referring to FIG. 4, another embodiment is shown where undulating bare spring wire 510 is integrated with mesh substrate 506 before electrospinning or dip coating. The apex portions at one end of the bare spring are interwoven in mesh substrate 506. The apex portions are shown encircled and numbered 512a, 512b, and 512c. Stent elements can be provided as described in any of the embodiments disclosed herein.

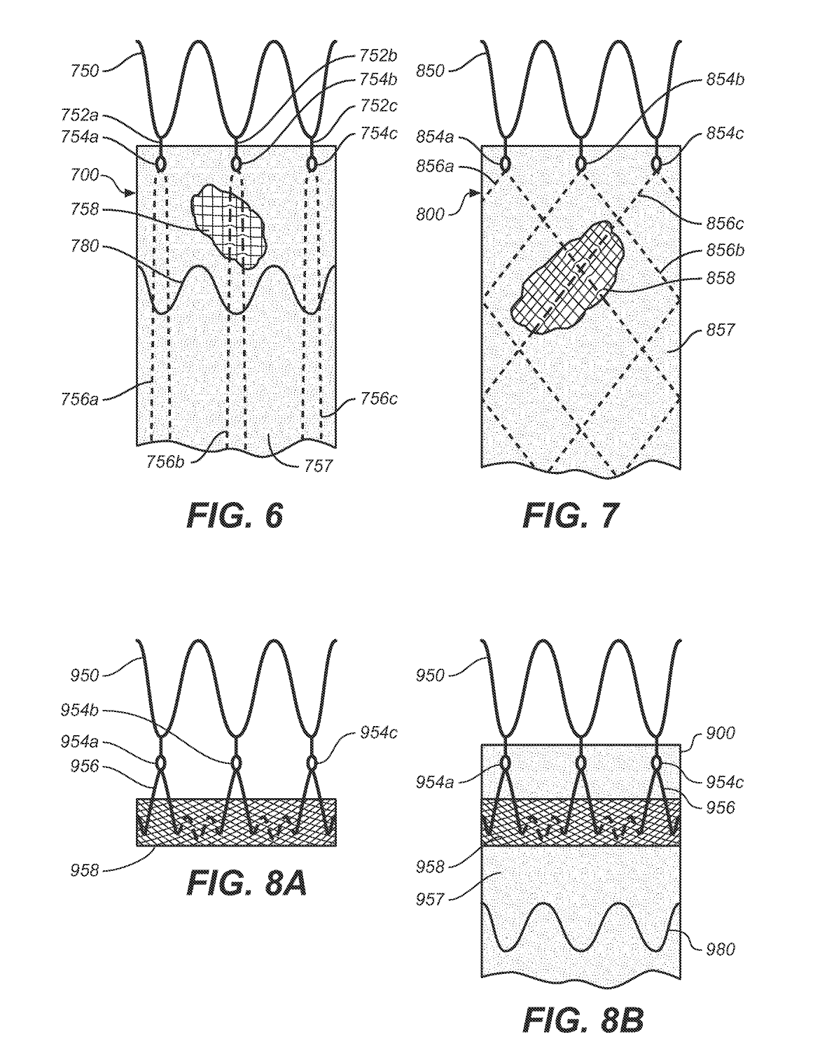

Referring to FIGS. 5A and 5B, integration of an anchor into a covered stent will be described. FIG. 5A depicts a known stent-graft anchor 600 having a barb support member or cage 602 with a plurality of barbs 604a,b,c,d extending from or secured to end portions along one side thereof. Wires or posts 606a,b,c,d having eyelets or loops 608a,b,c,d at their free ends extend from or are attached to the other side of the cage. Referring to FIG. 5B, the anchor is secured to covered stent 650 having one or more stents 652 integrated therein using a substrate as described above. The anchor is secured to the substrate with a plurality of high strength filaments 654a,b . . . n that are looped through eyelets 608a,b . . . n. The filaments, each of which can be a high strength fiber, are interwoven in the substrate along a portion of the length of the substrate such as shown in detail in FIG. 6 before the stent, substrate and eyelets are encapsulated in a polymer by way of, for example, an electrospinning or dip coating process to provide a tubular covered stent that is impervious to blood flow through the tubular cover. The substrate can run the entire length of the covered stent in this embodiment or any other embodiment described herein and the filaments can be interwoven along the entire length of the substrate to maximize filament incorporation or integration with the cover or polymeric membrane 656, which can comprise, for example, any of the materials described above. However, the substrate need not run the entire length of the covered stent and the filaments need not extend the entire length of the substrate. In another embodiment, the free ends of the filament can be adhered, tied otherwise secured to the substrate. Anchor 600 and the bare springs described below typically are attached to the proximal end of the covered stent, which is the end closest to the heart by reference to blood flow path when the covered stent is positioned in situ.

Referring to FIG. 6, another covered stent embodiment 700 is shown where a bare coil spring 750 with posts 752a,b,c . . . n extending therefrom and including eyelets 754a,b,c . . . n is secured to the covered stent in the same manner as anchor 600 is secured to covered stent 650. Each of a plurality of high strength filaments 756a,b . . . n, each of which can be a high strength fiber, are passed through a respective eyelet and interwoven in the mesh substrate that extends the entire length and the entire circumference of the covered stent. Covered stent 700 is shown in partial section with a portion of polymeric layer or cover 757 removed to show a portion of substrate 758 in an enlarged manner to illustrate filament 756b interwoven therein. Substrate 758 has a plurality of interwoven threads that can be interlaced like the warp and weft of a woven fabric. It should be understood that mesh patterns in all of the embodiments described herein are interchangeable and further that other patterns can be used as the illustrative embodiments are provided for the purposes of example and not to limit the scope of possible options. Covered stent 700 also includes one or more stents 780 which can have the same configuration as stents 202a-c and can be interwoven into mesh substrate 758 in the same manner as stents 202a-c are interwoven into braid-type mesh substrate 206.

Referring to FIG. 7, another covered embodiment 800, which is the same as covered stent 700 with the exception that the securing filament pattern differs. Bare spring 850 can have the same construction as bare spring 750 with eyelets 854a,b,c . . . n through which high strength filaments 856a,b,c . . . n are passed. Filaments 856a,b,c . . . n are interwoven in substrate 858, which can have the same construction as substrate 758 or any other suitable substrate. Covered stent 800 is shown in partial section with a portion of polymeric layer or cover 857 removed to show a portion of substrate 858 in an enlarged manner to illustrate filaments 856a and 856c interwoven therein. In the illustrative embodiment, each filament has one portion that extends in a clockwise helical direction and another portion that extends in a counterclockwise helical direction. The filaments in this embodiment can improve load distribution from the eyelets. Although not shown, covered stent 800 also includes one or more stents, which can have the same configuration as stents 202a-c and can be interwoven into mesh substrate 858 in the same manner as stents 202a-c are interwoven into braid-type mesh substrate 206.

Referring to FIGS. 8A and 8B, another covered stent embodiment 900 according to the invention is shown, where FIG. 8A illustrates an anchor and attachment component of covered stent 900 and FIG. 8B illustrates the anchor and attachment component integrally formed in the covered stent using electrospinning or dip coating techniques. Referring to FIG. 8A, bare spring 950 has the same construction as bare spring 750 and includes eyelets 954a,b,c . . . n through which a single high strength filament 956 is passed. Filament 956, which can be a high strength fiber, is interwoven in annular substrate 958, which can have the same construction as substrate 758 or any other suitable substrate. Annular substrate 958 can then be coupled to a tubular substrate such as substrate 206. Covered stent 800 also includes one or more stents 980, which can have the same configuration as stents 202a-c and can be interwoven into the mesh substrate to which substrate 958 is coupled in the same manner as stents 202a-c are interwoven into braid-type mesh substrate 206. The stent, bare spring, and substrate assembly is then encapsulated with a polymer using any suitable process such as electrospinning or dip coating.

Although not shown, any of the covered stents described herein can have a bifurcated configuration suitable for treating abdominal aortic aneurysms.

Any feature described in any one embodiment described herein can be combined with any other feature or features of any of the other embodiments or features described herein. Furthermore, variations and modifications of the devices and methods disclosed herein will be readily apparent to persons skilled in the art.

What is claimed is:

1. A covered stent suitable for placement in a lumen in a human body comprising:
a substrate;
a stent adapted to be placed in a lumen of a human body, said stent having portions interwoven in said substrate; and
encapsulation encapsulating said substrate and the portions of said stent interwoven in said substrate and forming a tubular graft member.

2. The covered stent of claim 1 wherein said substrate has a tubular shape.

3. The covered stent of claim 1 wherein said substrate extends less than the entire length of the tubular graft member.

4. The covered stent of claim 1 wherein said substrate extends the entire length of the tubular graft member.

5. The covered stent of claim 1 wherein said substrate comprises mesh material.

6. The covered stent of claim 5 wherein said encapsulation extends through the mesh material.

7. The covered stent of claim 1 wherein said encapsulation comprises polymeric material.

8. The covered stent of claim 7 wherein said encapsulation is formed through an electrospinning process.

9. The covered stent of claim 7 wherein said encapsulation is formed through s a dip coating process.

10. The covered stent of claim 1 including a plurality of said stents where each of said stents is interwoven in said substrate.

11. The covered stent of claim 1 wherein said tubular graft forms a wall that is impervious to blood flow therethrough.

12. The covered stent of claim 1 further including an anchor member and at least one filament, said filament extending through a portion of said anchor member and being interwoven in said substrate.

13. The covered stent of claim 1 wherein said anchor member has barbs extending therefrom.

14. The covered stent of claim 1 further including a bare spring and at least one filament, said filament extending through a portion of said bare spring and being interwoven in said substrate.

15. A covered stent suitable for placement in a lumen in a human body comprising:
a substrate;
a plurality of stents adapted to be placed in a lumen of a human body, said stents having portions interwoven in said substrate; and
a tubular polymeric member covering said stents and extending through at least a portion of said substrate.

16. The covered stent of claim 15 wherein said substrate comprises mesh.

17. The covered stent of claim 15 further including an anchor and a filament secured to the anchor and said substrate.

18. The covered stent of claim 15 wherein said anchor comprises a bare spring element.

19. The covered stent of claim 15 wherein said anchor has a barb extending therefrom.

20. A method of making a covered stent comprising:
interweaving a wire though a substrate to form a tubular member;
encapsulating the tubular member to form a covered stent having a tubular cover suitable for placement in a lumen of a human body.

21. The method of claim 20 wherein encapsulating comprises electrospinning a polymer over the stent and substrate.

22. The method of claim 20 wherein encapsulating comprises dipping the stent and substrate in polymeric material.
